(19) United States
(12) Patent Application Publication
(10) Pub. No.: US 2012/0160249 A1
(43) Pub. Date: Jun. 28, 2012

(54) TONGUE ADVANCEMENT DEVICE FOR REDUCING OBSTRUCTIVE SLEEP APNEA CONDITION

(76) Inventors: Thomas B. Thomason, Santa Fe, NM (US); Ronald G. Garrett, Clovis, NM (US)

(21) Appl. No.: 13/336,930

(22) Filed: Dec. 23, 2011

Related U.S. Application Data
(60) Provisional application No. 61/427,149, filed on Dec. 24, 2010.

(51) Int. Cl. A61F 5/56; A61C 5/14

(52) U.S. Cl. 128/848

(57) ABSTRACT

A tongue advancement device according to various embodiments can include at least one first magnet provided in a mouthpiece and a tongue implantation device. The at least one first magnet comprises a substantially flat surface that aligns with a peripheral edge of the mouthpiece, such that the at least one first magnet does not extend from the mouthpiece. The tongue implantation device, such as a tongue piercing, includes a second magnet that magnetically interacts with the at least one first magnet to retain the tongue of the user in a forward position when the mouthpiece is worn. The device can also include a tongue cover consisting of a flexible tubular body having an opened end, a closed end and a magnet attached to the closed end. The tongue cover may be configured as a tongue sleeve or a tongue gripper based upon the principals of the Chinese finger trap.

### REFERENCE TO RELATED APPLICATION

This application is a continuation-in-part of provisional application Ser. No. 61/427,149, filed Dec. 24, 2010, which is incorporated by reference herein.

### FIELD OF THE INVENTION

The present teachings relate to devices and method for moving and restraining the tongue to keep a person's airway open to permit air flow into the lungs to facilitate breathing during sleep and to treat sleep-related breathing disorders, such as sleep apnea and snoring.

### INTRODUCTION

Millions of people snore while sleeping and approximately 20% of them suffer from Obstructive Sleep Apnea (OSA). OSA is a serious medical condition that is difficult to treat effectively. The condition is marked by a partial or complete closure of the upper airway during sleep. Thereby, restricting or completely stopping the breathing process. Breathing is fundamental to maintaining human life. Some of our most fundamental reflexes involve breathing and maintaining an open airway. Anything that interferes with breathing may constitute a life-threatening situation. For example, an unconscious or semi-conscious person who develops a partially or fully blocked airway may die if the airway is not open enough to allow enough air to reach the lungs. The tongue is a muscle, which is normally maintained in a somewhat forward position under autonomic nervous system control. This control, along with control of breathing and heart rate, is affected by the medulla section of the brain. The autonomic control may or may not be maintained even in a person who is sleeping, semi-conscious, unconscious or under heavy sedation. Certain conditions such as severe trauma or obstruction can compromise these autonomic reflexes. If a person's head is oriented with the mouth pointing upwards and the tongue is too relaxed, the tongue can move to close the person's throat, reducing or even closing the person's airway. If this severe closure condition is not corrected within several minutes, preferably within one or two minutes, this can cause a medical emergency, and if the condition is not corrected within a very few minutes, it can lead to death. Blocking the airway for about two minutes can begin to reduce oxygen saturation in the brain. Maintaining the blockage for four to six minutes can begin to cause brain damage. Blocking the airway for ten more minutes can lead to irreversible brain damage.

In a given night, the number of involuntary breathing pauses or “apneic events” in OSA patients may be as high as 20 to 60 or more per hour. These breathing pauses are almost always accompanied by snoring between apnea episodes. Thus, OSA can also be characterized by choking sensations. Snoring typically occurs when a person sleeps on his back. This snoring sound is created by ingress and egress of air vibrating against the uvula and/or soft palate, the uvula is a small conical fleshy tissue that hangs from the middle of the soft palate. The size of a normal airway is about the diameter of a pencil. In OSA patients, the muscles of the soft palate at the base of the tongue and the uvula relax and sag. This obstructs the airway and makes breathing labored and noisy. Those with OSA stop breathing in their sleep, often hundreds of times during the night. Usually OSA occurs when the throat muscles and tongue relax during sleep and partially block the opening of the airway. Sleep apnea also can occur in obese people when an excess amount of tissue in the airway causes it to be narrowed.

There are several choices of care for this problem with the most common and well known being a Continuous Positive Airway Pressure device, commonly known as CPAP. CPAP acts as a pneumatic splint which literally blows open the upper airway during sleep such that the tendency for the upper airway to collapse is either eliminated or reduced. CPAP machines have many drawbacks. One in particular, the nasal assembly of the CPAP is structured such that stability forces applied by the headgear of the CPAP are distributed to the back of the patient's head, the patient's cheeks, and the patient's upper lip to maintain the nasal assembly in an air tight condition on the patient's face. The nasal assembly includes a nozzle assembly structured to apply sealing forces to nasal passages of the patient's nose. Thus, the CPAP machine is expensive, noisy, intrusive for the patient and difficult to maintain.

There are a variety of surgeries to treat OSA, some approaches work better than others. Surgeries are not guaranteed and they are very expensive and the patient suffers initial discomfort. Some patients with OSA may need surgery. Although several surgical procedures are used to increase the size of the airway, none of them are without risks. More than one procedure may need to be performed before the patient realizes any benefits. Some of the more common procedures include removal of adenoids and tonsils, nasal polyps or other growths, or other tissue in the airway and correction of structural deformities.

It may be desirable to provide a system and method which alleviate the claustrophobic effects which could be associated with existing sleep apnea CPAP. It may also be desirable to provide a system and method that is less intrusive than a traditional CPAP sleep apnea mask and does not require the risk of surgery. It also may be desirable to provide a system and method that is relatively efficient and simple in terms of design and implementation.

### SUMMARY

The present invention may satisfy one or more of the above-mentioned desirable features. Other features and/or advantages may become apparent from the description which follows.

A tongue advancement device according to various exemplary embodiments can include a mouthpiece, at least one first magnet and a tongue implantation device. The mouthpiece is configured to fit within the mouth of a user such that the mouthpiece fits over at least a portion of the teeth of the user. The at least one first magnet is provided in the mouthpiece and comprises a substantially flat surface that aligns with a peripheral edge of the mouthpiece, such that the at least one first magnet does not extend from the mouthpiece. The tongue implantation device includes a second magnet that magnetically attracts to the at least one first magnet such that a magnetic attraction between the at least one first magnet and the second magnet retains the tongue of the user in a forward position when the mouthpiece is worn in the mouth.

A tongue advancement device in other embodiments can include a mouthpiece, at least one first magnet, a tongue cover and at least one second magnet. The mouthpiece is configured to fit within the mouth of a user such that the mouthpiece fits over at least a portion of the teeth of the user. At least one first magnet is provided in the mouthpiece. The tongue cover comprises a flexible tubular body having an opened end and a closed end. The opened end is configured to receive the tongue such that the flexible tubular body encircles and conforms to the shape of the tongue when inserted therein. At least a portion of the tubular body includes a permeable surface to enable fluids to pass through and contact the tongue. The at least one second magnet is provided at the closed end of the tongue cover to magnetically interact with the at least one first magnet via a magnetic attraction such that a force of the magnetic attraction between the at least one first magnet and the second magnet retains the tongue of the user in a forward position when the mouthpiece is worn in the mouth.

Various embodiments of the tongue cover may comprise a tongue sleeve including a perforated surface. The tongue sleeve may include a fastening mechanism, such as a pair of ear straps, a unitary headband or an adjustable headband, to assist with securing the tongue cover onto the user's tongue. Other embodiments of the tongue cover may comprise a mesh network configuration or a tongue gripper consisting of a plurality of cords configured to form a Chinese finger trap configuration.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the invention.

### BRIEF DESCRIPTION OF THE DRAWINGS

The skilled artisan will understand that the drawings described below are for illustrative purposes only. The drawings are not intended to limit the scope of the present teachings in any way.

FIG. 1A illustrates an exemplary embodiment of a mouthpiece and a tongue implantation device of a tongue advancement device detached from each other;

FIG. 1B illustrates an exemplary embodiment of a mouthpiece, a dental mold used to form the mouthpiece and a tongue implantation device detached from the mouthpiece;

FIG. 2A illustrates a tongue implantation device including a magnet and a stud extending through the user's tongue and attracted towards a magnet included within the mouthpiece;

FIG. 2B illustrates a tongue implantation device magnetically attached to a mouthpiece;

FIG. 2C illustrates a tongue implantation device magnetically attached to a mouthpiece positioned on a dental mold;

FIG. 3A illustrates a user inserting an upper mouthpiece;

FIG. 3B illustrates a user inserting a lower mouthpiece;

FIG. 4 illustrates a tongue implantation device including a magnet and a stud extending through a user's tongue;

FIG. 5 illustrates a tongue implantation device including a magnet and a stud extending through a user's tongue;

FIG. 6 illustrates a top portion of a tongue implantation device;

FIG. 7 illustrates a side view of a tongue implantation device;

FIG. 8 illustrates a tongue implantation device attached to an overlay;

FIG. 9 illustrates a side view of a tongue implantation device attached to an overlay;

FIG. 10 is another exemplary embodiment of the tongue advancement device comprising a tongue sleeve;

FIG. 11A illustrates the tongue sleeve of FIG. 10 including a pair of ear straps;

FIG. 11B illustrates the tongue sleeve of FIG. 10 including an adjustable headband;

FIG. 11C illustrates the tongue sleeve of FIG. 10 including a unitary headband;

FIG. 12A is another exemplary embodiment of the tongue advancement device comprising a tongue gripper configured based upon a Chinese finger trap; and

FIG. 12B is illustrates the tongue gripper in FIG. 12 with the user's tongue inserted therein.

### DETAILED DESCRIPTION OF VARIOUS EMBODIMENTS

Reference will now be made to various embodiments, examples of which are illustrated in the accompanying drawings. However, these various exemplary embodiments are not intended to limit the disclosure. On the contrary, the disclosure is intended to cover alternatives, modifications, and equivalents.

Throughout the application, description of various embodiments may use “comprising” language, however, it will be understood by one of skill in the art, that in some specific instances, an embodiment can alternatively be described using the language “consisting essentially of” or “consisting of.”

For purposes of better understanding the present teachings and in no way limiting the scope of the teachings, it will be clear to one of skill in the art that the use of the singular includes the plural unless specifically stated otherwise. Therefore, the terms “a,” “an” and “at least one” are used interchangeably in this application. Unless otherwise indicated, all numbers expressing quantities, percentages or proportions, and other numerical values used in the specification and claims, are to be understood as being modified in all instances by the term “about.”

Unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained.

Various embodiments of the tongue advancement device desired herein enable moving and restraining of the tongue to keep a person's airway open to permit air flow into the lungs to facilitate breathing during sleep and to treat sleep-related breathing disorders, such as sleep apnea and snoring. Various embodiments of the tongue advancement device desired herein enable a non-medical professional to quickly and easily install and remove the device. Various embodiments of the device enable control of the user's tongue and improve snoring or sleep apnea by holding the tongue forward during sleep. Various embodiments of the tongue advancement device enable the user to selectively retain the tongue at different positions to control the flow of air into the user's mouth. Various embodiments include an upper mouthpiece, which can be placed on the user's upper teeth. Some embodiments include a lower mouthpiece, which can be placed on the user's lower teeth. Various embodiments of the tongue advancement device can be inserted to cover only select portions of the user's mouth.

Various embodiments are directed towards reducing snoring and alleviating OSA by controlling the flow of air into the user's mouth and providing more regular breathing cycle. In comparison to conventional devices, various embodiments of the tongue advancement device described herein provide a lightweight and comfortable device so that OSA patients are not tempted to discard uncomfortable ventilation devices while sleeping. Various embodiments enable installation and placement of the device in an outpatient setting, with minimal loss of time from work or daily activities.

In various embodiments, the operation of the device may be relatively simple and robust, and may enable retention of a user's tongue without the attachment of external mechanical devices or equipment that require electricity. Placement does not require hospitalization of the user. The device is small portable and easily replaceable. Thus allowing relatively easy placement by minimally trained personnel.

Various embodiments can be employed by users having a pierced tongue. Some embodiments can be employed by users not having pierced tongues.

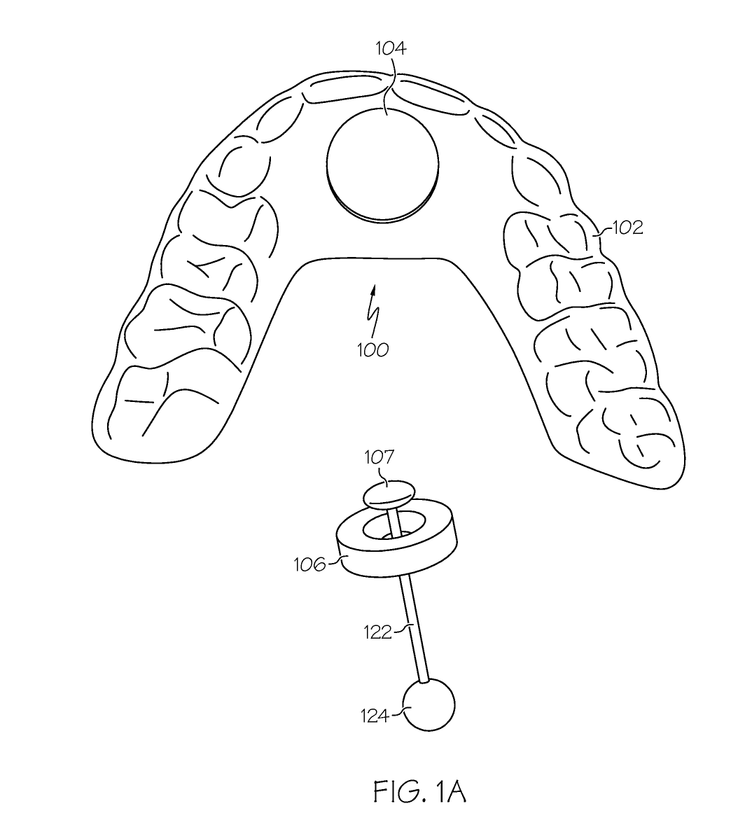

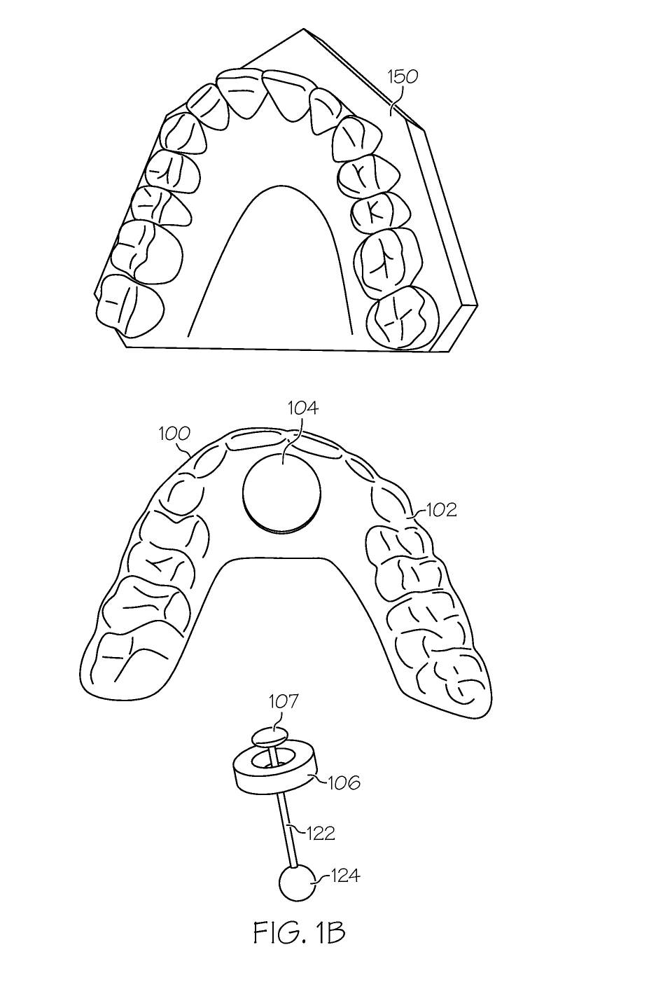

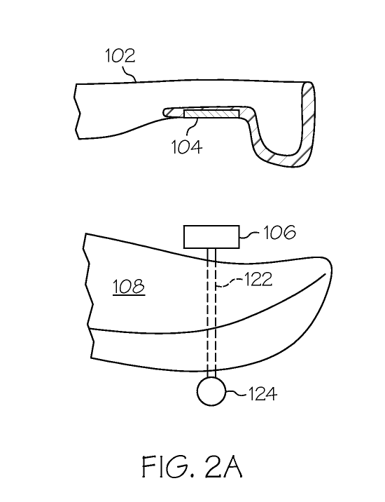

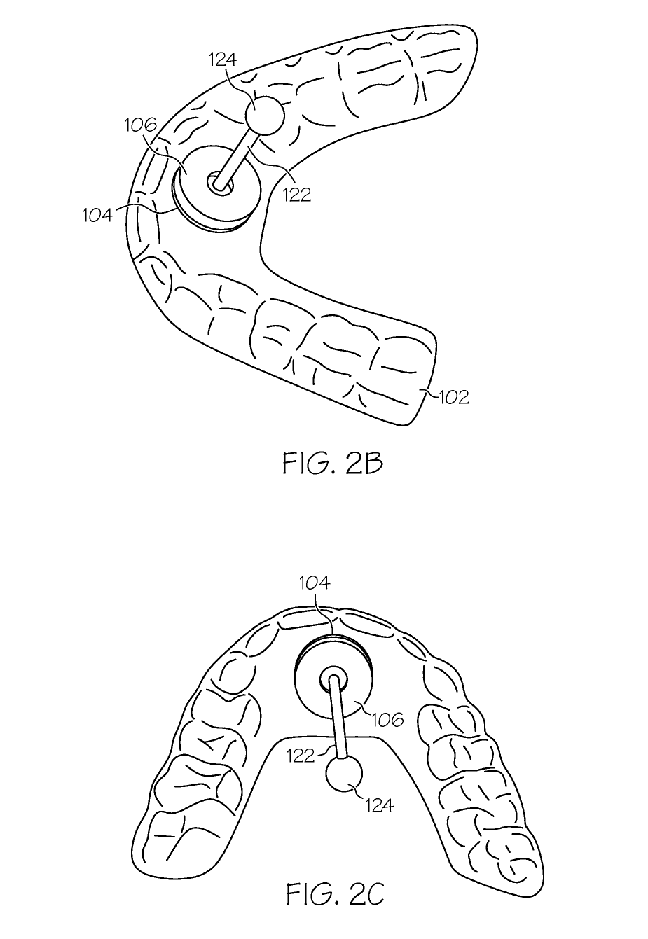

An exemplary embodiment of the tongue advancement device 100 that can be used, for example, to move and restrain the tongue to keep a person's airway open to permit air flow into the lungs to facilitate breathing during sleep and to treat sleep-related breathing disorders, such as sleep apnea and snoring, is illustrated in FIG. 1A. The device 100 involves the movement of the tongue 108 using the forces of magnets. The device 100 can include a plastic removable mouthpiece 102 that can be removably inserted into the user's mouth to hold one or more upper magnets 104 in alignment with one or more lower magnets 106 inserted through the user's tongue 108 (FIG. 2A). In FIGS. 2A-2C, the magnets 104, 106 are placed on the mouthpiece 102 and through the tongue 108, respectively, in such a manner to employ the attraction characteristics of a magnetic field. The device 100 can be inserted by the user at night so that these magnetic forces are employed to move the tongue 108 into a forward position while the user sleeps.

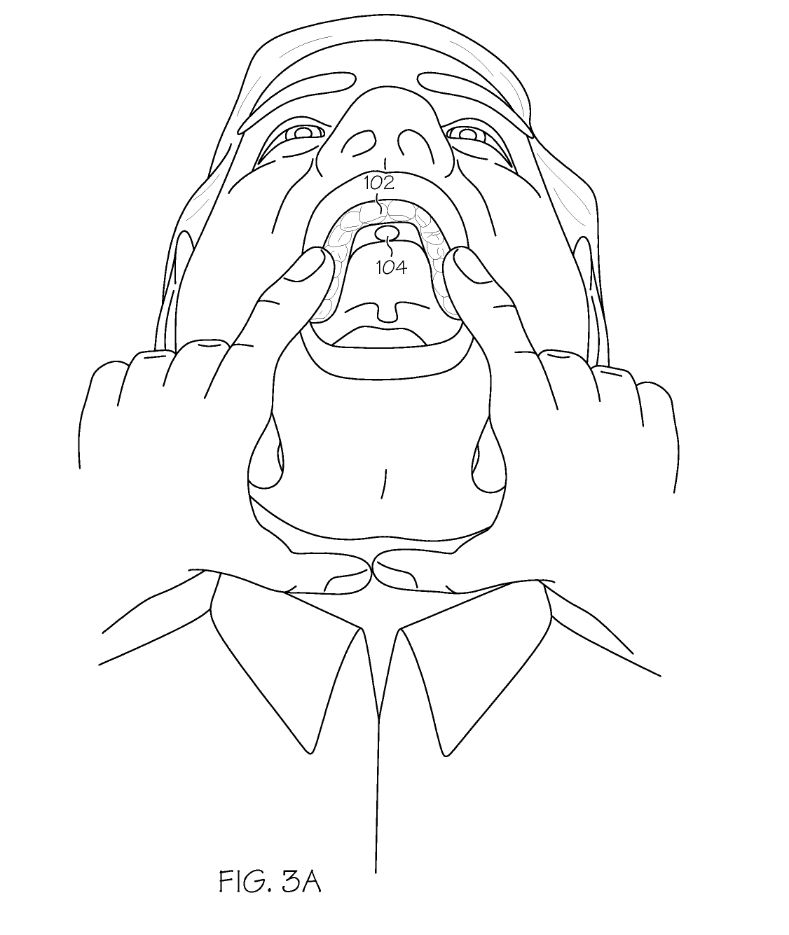

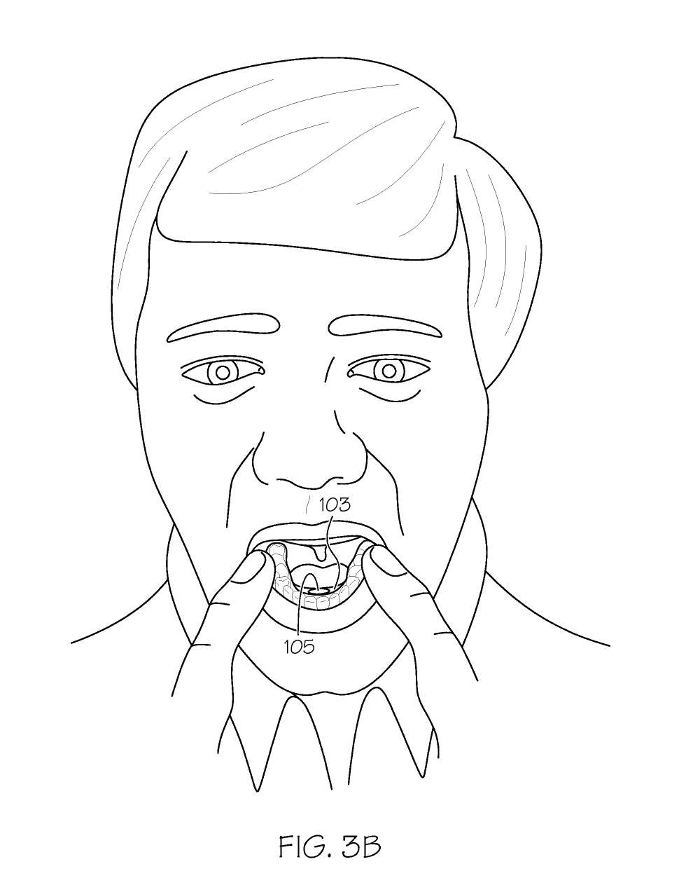

As shown in FIGS. 1A, 1B and 3A, the mouthpiece 102 can be configured having a one piece construction that covers and fits over the user's teeth. The mouthpiece 102 can be constructed similar to devices used for teeth whitening or post orthodontia treatment to maintain tooth position. Any suitable moldable plastic may be used for the plastic mouthpiece 102. A suitable material may be a heat-sensitive synthetic such as ethylene vinyl acetate, like those used for football mouth guards, which softens sufficiently in hot water to mold the plastic material to the configuration of the user's mouth and teeth. Thus, the device provides a mouthpiece that is relatively inexpensive to fabricate, simple to apply, and effective in use. Alternatively, a dental mold 150 may be employed to fabricate the plastic mouthpiece 102. In some embodiments, the mouthpiece 102 can be configured to extend continuously from tooth to tooth in the tooth region. In other embodiments, the mouthpiece 102 can be configured to extend from a select number of teeth in the tooth region such that the mouthpiece covers a portion of the tooth region. The mouthpiece 102 may vary both in size and shape, depending on the user's oral topography.

As illustrated in FIGS. 1A, 2A and 2B, the mouthpiece 102 may have at least one small upper magnet 104 having a magnetic force sufficiently strong to magnetically attract and remain in contact with lower magnet 106. Magnet 104 can be placed on the back side of mouthpiece 102 such that it does not directly connect the user's tongue 108, which enhances the user's comfort when wearing the device while asleep. Using a fastening mechanism (not shown), magnet 104 may be attached anywhere on the mouthpiece 102, but preferably in a forward position of the mouthpiece 102, just behind the area where the front middle teeth are positioned (FIG. 3A). The fastening means may include thermoplastic molding, riveting, screwing, and gluing. Magnets 104, 106 can vary in configurations, including round, square or rectangular shapes. Magnet 104 can be formed within the mouthpiece 102 as a single magnet or consists of multiple magnets embedded within the mouthpiece during the manufacturing process or releasably attached to the back side of the mouthpiece 102. In some embodiments, the magnet 104 may include a magnetic strip embedded within or removably attached to the mouthpiece 102. In the embodiments including multiple magnets, each magnet 104 may be strategically placed in several different locations within or onto the mouthpiece 102 such that, when in use, the user has the option to selectively place the tongue 108 at a specific location to enhance the user's comfort and effectiveness of the device during sleep. In some embodiments, the magnetic strip may be applied such that it extends longitudinally along and covers the entire forward section of the mouthpiece which covers the upper teeth. In the embodiments directed to magnet 104 being removably attached to the mouthpiece 102, the user may easily interchange magnet 104 with another magnet having a different magnetic strength. Furthermore, the device 100 permits selective adjustment of the forward position of the user's tongue relative to the mouthpiece such that the tongue may be comfortably positioned and retained.

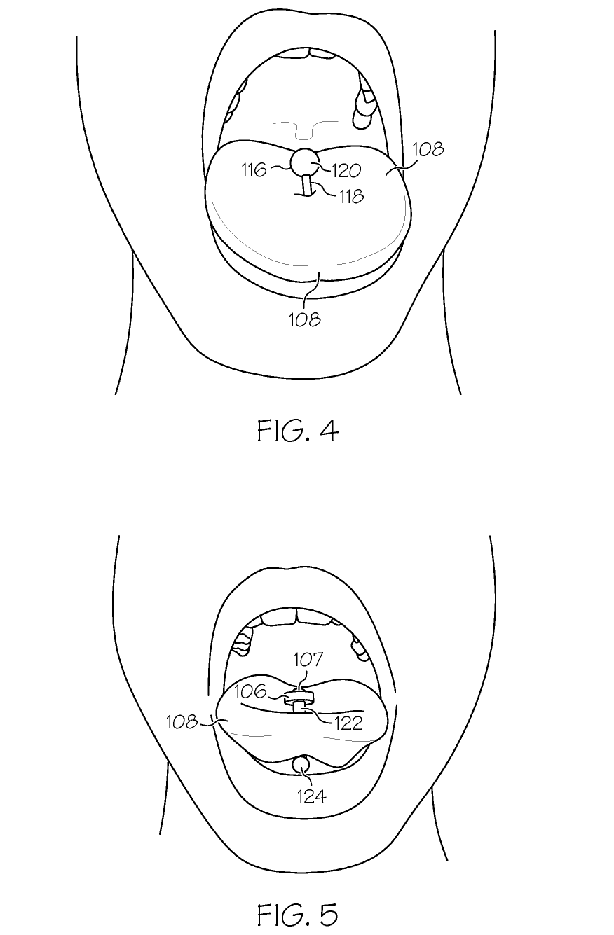

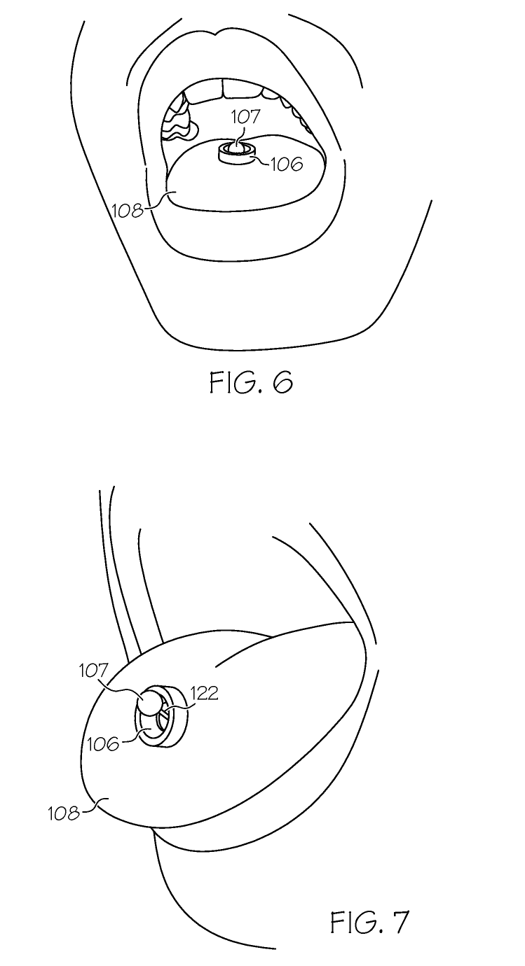

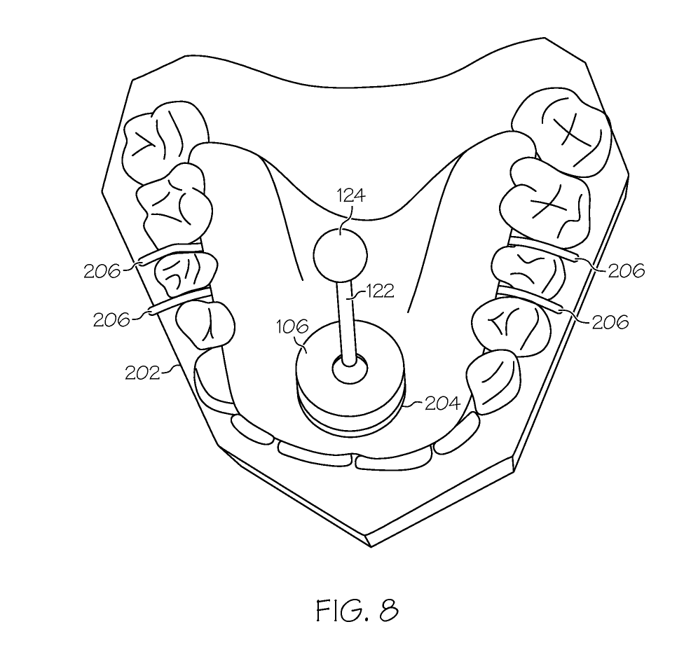

FIG. 4 illustrates a user wearing a decorative tongue piercing 116 extending through a hole in the user's pierced tongue 108 by connecting one end of a stud 118 to a decorative element 120, such as a ball and removably connecting an opposing end of the stud 118 to the underside of the tongue 108 by way of a fastening mechanism (not shown). To use device 100, the user removes the decorative element 120 along with stud 118 from the hole in his tongue 108. Then, as shown in FIG. 5, through the hole in his tongue, the user inserts stud 122 attached at one end to retaining ball 107 and attached at the other end to a fastening mechanism 124. In the embodiment shown in FIGS. 5-7, the retaining ball 107 is shown as a separate component that securely holds magnet 106 within the user's mouth. In an alternate embodiment, the magnet 106 can be configured as a single component that connects directly to stud 122, without a retaining ball. Retaining ball 107 eliminates or considerably reduces the risk that the magnet 106 will come loose from stud 122 during use. In an alternative embodiment to use device 100, the user can merely remove the decorative element 120 and replace the decorative element 120 with magnet 106. Thus, in various embodiments, stud 122 may or may not be replaced during the replacement of decorative element 120 with magnet 106.

As shown in FIGS. 2A-2C, magnets 104 and 106 possess poles of opposite polarity to attract each other with a magnetic force. Both magnets 104 and 106 are selected having sufficient agnetic forces to prevent misalignment in order to maintain the desired tongue position during use and to prevent the tongue from becoming dislodged and falling into the user's airway while asleep. Magnets 104, 106 described thus far may be made of any suitable, permanent magnetic material, including rare earth. The fabrication and polarization of such magnets are well known in the magnet art.

During use, the user inserts the mouthpiece 102 to fit over the upper teeth such that the upper magnet 104 is positioned slightly behind the area of the front middle teeth (FIG. 3A), and then the user uses stud 122 to attach the lower magnet 106 to the tongue 108. Upon insertion of the device 100 into the user's mouth, due to the placement of the upper magnet 104 attached to the mouthpiece 102 and the lower magnet 106 inserted through the user's tongue piercing, the magnetic attraction between magnet 104 and magnet 106 will encourage the user's tongue forward to keep the user's airway open during sleep (FIG. 2A). When the user awakes, the user can remove both the mouthpiece 102 and the lower magnet 106 stud and can place a clear plastic mouthpiece (not shown) into the pierced tongue, if desired, to maintain patency of the hole in the tongue and to present a more professional appearance during the day, for example, while at work.

Essentially, use of the device 100 is an inexpensive process, which involves a few simple steps. First, the mouthpiece 102 is formed. The mouthpiece can be made by using a heat-sensitive synthetic plastic forming process. All the components of the device may be sold as a kit, which includes the heat-sensitive synthetic plastic. Alternatively, the user may use any suitable consumable, off-the-shelf heat moldable tray, similar to those used to form mouth guards or bleaching trays. Another alternative is that the mouthpiece 102 can be made by a dental professional by creating a mold that is an exact duplicate of the user's upper teeth. Most likely, the dental mold will provide a mouthpiece having a better custom fit for the user. Next, a fastening means (not shown) is used to attach magnet 104 to the back side of the mouthpiece 102. The fastening means may include, for example, thermoplastic molding, riveting, screwing and gluing. Finally, the lower magnet 106 is attached to the user's tongue. If the user's tongue is pierced, the user can remove any existing piercing 116 and replace it with lower magnet 106. An embodiment describing use of the device when the user's tongue is not pierced is provided below. In comparison, using device 100 and its process will cost the user approximately $200, whereas a CPAP machine would cost approximately $2000.

In the preferred embodiment, the upper mouthpiece 102 is placed on the user's upper teeth, because the distance to the back of the throat and the upper teeth remain relatively unchanged with respect to the lower jaw position, which commonly falls posteriorly due to the relaxation of the muscles of the jaw when the user sleeps. In lieu of or in conjunction with the upper mouthpiece 102, as shown in FIG. 3B, the device 100 may include a lower mouthpiece 103 formed having a segment that corresponds to a portion or the user's entire lower teeth contour. Similar to the upper mouthpiece 102, the lower mouthpiece 103 may include one or more magnets 105 attached or formed therein. Some of the benefits of employing the lower mouthpiece 103 with the magnet are that it permits usage for users who are edentulous on the upper jaw and permits easier mouth breathing for users whose nasal airway have been compromised. The lower mouthpiece 103 may also cause less feeling of claustrophobia for users who are intolerant of the upper teeth mouthpiece device. Other benefits of the lower mouthpiece include that it may decrease the gag reflex that some user may experience with an upper device, and the lower device may cause less chance for choking in the cause of nausea or vomiting.

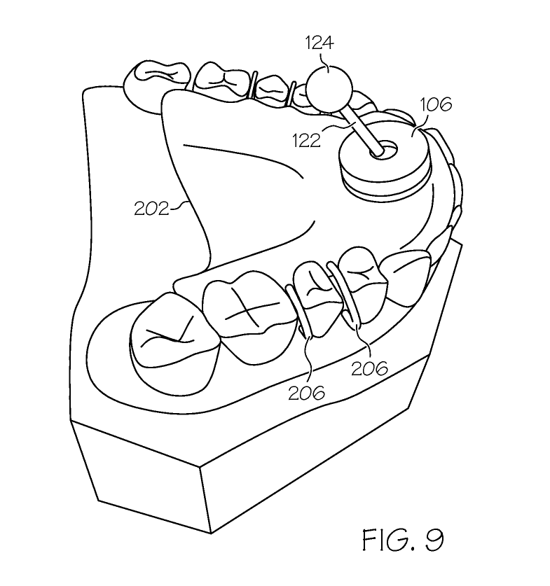

In lieu of a mouthpiece 102 that covers and fits over the contour of the user's teeth, as shown in FIGS. 1A, 1B, 3A, and 3B, the mouthpiece 202 in FIGS. 8-9 may include an overlay 204 comprising a pair of opposed spaced retaining and positioning wires 206 inserted therein. In some embodiments, the retaining wires 206 may be configured as clips that hold mouthpiece 202 in place. The retaining wires 206 can be made of metal, such as stainless steel. Of course, various modifications can be made to the present invention within the scope of the claims herein. FIGS. 8-9 show the overlay 204 as a palatal overlay attached to a cast molding. The palatal overlay 204 is molded to conform to the shape of the forward portion of the palatal cavity against the lingual or inside half of the upper teeth. For a lower mouthpiece, mouthpiece 202 may be adapted for the lower teeth by providing in place of the palatal overlay a gingival overlay molded to conform to the lingual side of the lower teeth. Both the palatal and gingival overlay can be formed using conventional orthodontic procedures. As shown in FIGS. 8-9, a magnet can also be included within the overlay 204 to attract and hold the lower magnet 106 of the magnetic tongue piercing when inserted into the user's tongue, as described above.

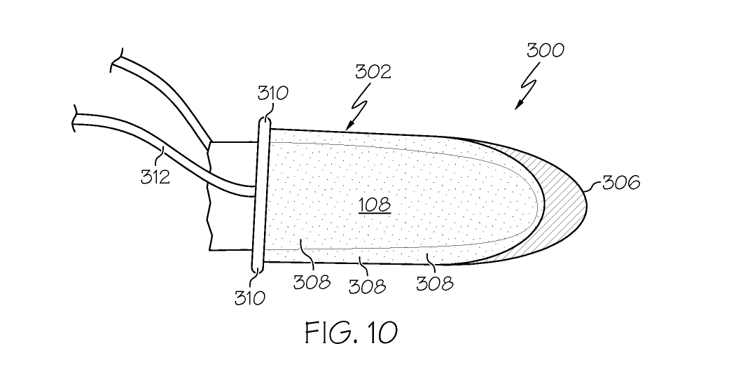

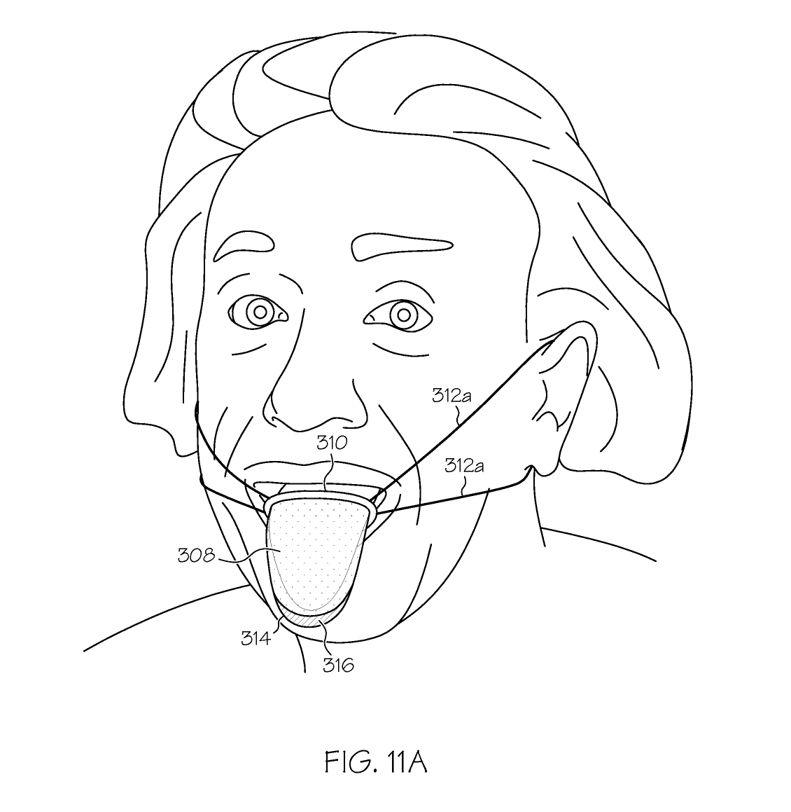

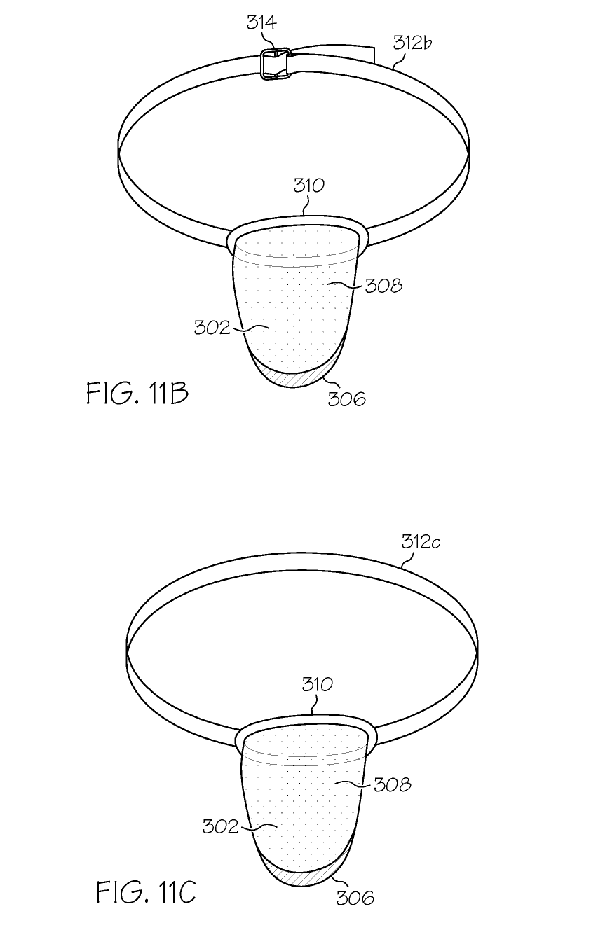

In an alternate embodiment, as shown in FIG. 10, the device 300 can be worn by a person without a pierced tongue by using a perforated tongue sleeve 302. The tongue sleeve 302 comprises a thin, elastic, perforated, substantially tubular membrane having a closed end and a supporting ring structure 310 at its open end. The tongue sleeve 302 may be made of a flexible water insoluble material, such as a polymer and may be approximately 3-5 inches to accommodate the average length of a human's tongue. The user either pulls or rolls the tongue sleeve 302 onto his tongue 108. Elastic ring 310 securely holds the tongue sleeve 302 onto the user's tongue 108 in a comfortable manner. A fastening mechanism 312 may be attached at the open end of the tongue sleeve 302 to further assist with holding the sleeve onto the user's tongue during sleep. The fastening mechanism 312 can be made of resilient rubber strings and attached to the left and right sides, respectively, of the tongue sleeve. The fastening mechanisms 312 are preferably attached to or integrally formed therein during the manufacturing of the tongue sleeve. In FIG. 11A, the fastening mechanism is shown as elastic ear straps 312a that extend over and fit comfortably and securely upon the user's ears. In FIG. 11B, the fastening mechanism is an elastic strap 312b including a buckle 314 that enables the strap to adjust around the user's head. In FIG. 11C, the fastening mechanism is shown having an elastic headband 312c configuration that stretches and extends around the user's head.

In some embodiments, the tongue sleeve 302 may be manufactured and sold as a standard, one-size-fit all consumable. In other embodiments, the tongue sleeve 302 may be made be manufactured and sold as a consumable having different sizes, for example, small, medium and large. Perforations 308 are provided in the tongue sleeve 308 to enable saliva to flow through the perforations and lubricate the tongue during use. It is generally accepted that during sleep the amount of salivary production substantially decreases. The tongue is sensitive and kept moist by saliva. The perforations 308 allow saliva to enter the sleeve and moisten the tongue to prevent the tongue from becoming dry or prevent the user from having a dry mouth sensation. A magnet 306 may be included at the closed end of the tongue sleeve 302 to provide a magnetic force that attracts magnet 104 and pull the tongue 108 forward, as described above. The magnet 306 may be embedded within the closed end of the tongue sleeve during the manufacturing process. In some embodiments, the tongue sleeve can be designed with a pocket 314 having an opening 316 for removably inserting the magnet 306 into the tongue sleeve to interchange the magnet, for example, with a magnet having a different strength. Magnet 306 can be formed within the tube sleeve 302 as a single magnet or consist of multiple magnets embedded within or removably attached to tongue sleeve 302. In some embodiments, magnet 306 may include a magnetic strip. In the embodiments including multiple magnets, each magnet 306 may be strategically placed in several different locations within or onto the tube sleeve 302. For example, magnet 306 may be placed along the side of the tube sleeve to enable the side portion of the tongue to be pulled and attracted to a magnet included in the upper retain or lower mouthpiece. Thus, this embodiment provides a device for retaining a tongue in a predetermined position when the user's tongue is not pierced.

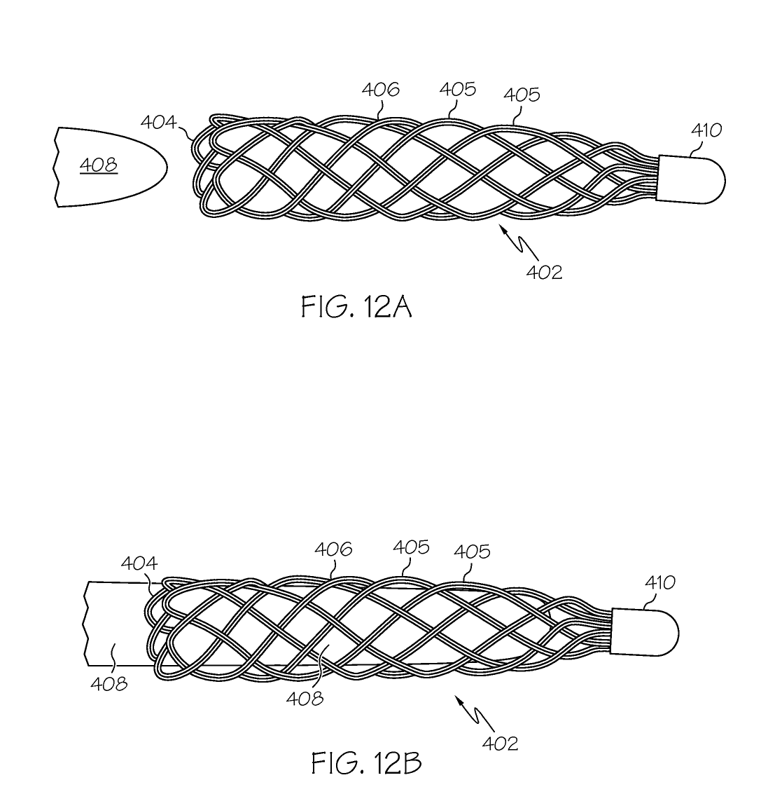

FIGS. 12A-12B illustrate another alternative embodiment of the device 400 that can be worn by a person without a pierced tongue by using a tongue gripper 402, designed based upon the principal of the well-known “Chinese finger trap.” The tongue gripper 402 comprises an open end portion 404, a tubular shaped open mesh 406, and a magnetic closure 410 which holds the mesh closed. The tongue gripper 402 can be designed having three or more braided cords 405. As shown, a plurality of cords are braided and intertwined together to form the tubular shaped open mesh 406. The cords 405 of the gripper can be constructed of thin, flexible water insoluble material, such as a polymer or fabric, and the magnet 410 can be selected having the properties as described in any of the above embodiment.

As shown in FIG. 12A, end portion 404 of the tongue gripper is wide and open to receive a tongue 408. During use, the open end 404 receives the user's tongue 408 such that it encircles and grips the tongue, as shown in FIG. 12B. The magnetic end 410 of the gripper 402 is then attached to the magnet 104 or 105 (FIGS. 3A and 3B) within the upper or lower retain, respectively. Due to the nature of the Chinese trap action of the meshed sleeve 406, the tongue will be held securely in the forward position as the user sleeps. The magnetic force between magnet 410 of the tongue gripper 402 and magnet 104 or 105 creates a traction into the mesh 406 such that the braided cords 405 move with respect to each other to prevent the tongue from falling backward. Any backward movement of the tongue creates a pulling force at the open end 404 causing the circumference of the mesh 406 to shrink and tighten around the tongue and holds the tongue in the forward position. Removal of the tongue gripper 402 from the tongue is easily accomplished by detaching the magnet 410 from either the upper or lower magnet 104, 105 which releases the tension of the braids 405 to permit the tongue to be gently removed. Furthermore, the uniform and evenly distributed loading of the gripping forces of the mesh 406 is such that it holds the tongue without damage. The device 400 does not interfere with blood circulation in the tongue, and the open mesh net configuration allows saliva to moisten the tongue to prevent the tongue or mouth from becoming dry.

The tongue advancement device allows the tongue to be held forward; thus, preventing posterior displacement and collapse of the upper oral airway. This is achieved by using a relatively simple, reversible, removable, patient maintainable, and adjustable strength device. The device is relatively inexpensive, portable and does not require a source of electricity to be functional. The device can be worn when the patient sleeps and removed during the waking hours. It will be apparent to those skilled in the art that various modifications and variations can be made to the tongue advance device and method of the present disclosure without departing from the scope of its teaching. By way of example, tongue advancement devices in accordance with the present teachings, may include a tongue stud made of multiple materials. In some embodiments, the tongue stud can be either straight or curved. In various embodiments, the mouthpiece can be made of either a soft or hard material. Rather than using a magnet, the device can employ a hook and loop fastener arrangement to attach the user's tongue to the mouthpiece. In other embodiments, a surgical operation can be performed to permanently implant a magnet into the tongue at a location which is not visible. A magnet can also be permanently placed into the bone of the upper jaw. For edentulous patients, implants can be provided as an option to allow placement of the mouthpiece.

Other embodiments of the disclosure will be apparent to those skilled in the art from consideration of the specification and practice of the teachings disclosed herein. It is intended that the specification and examples be considered as exemplary only.

What is claimed is:

1. A tongue advancement device, comprising:
a mouthpiece configured to fit within the mouth of a user such that the mouthpiece fits over at least a portion of the teeth of the user;
at least one first magnet provided in the mouthpiece, wherein the at least one first magnet comprises a substantially flat surface that aligns with a peripheral edge of the mouthpiece, such that the at least one first magnet does not extend from the mouthpiece; and
a tongue implantation device including a second magnet that magnetically attracts to the at least one first magnet to retain the tongue of the user in a forward position when the mouthpiece is worn in the mouth.

2. The device of claim 1, wherein the tongue implantation device comprises a stud extending through a pierced tongue of the user.

3. The device of claim 1, wherein the second magnet is removable and interchangeable with a decorative tongue piercing element.

4. The device of claim 1, wherein the at least one first magnet is removably attached to the mouthpiece such that the at least one first magnet is interchangeable with another magnet having a different magnetic strength.

5. The device of claim 1, wherein the at least one first magnet comprises a plurality of magnets provided within the mouthpiece, wherein each magnet is placed at different locations within the mouthpiece for enabling the user to selectively retain the tongue at one of the different locations of the magnets.

6. The device of claim 1, wherein the at least one first magnet comprises a magnetic strip.

7. The device of claim 1, wherein the mouthpiece comprises an upper mouthpiece or a lower mouthpiece.

8. The device of claim 7, wherein the mouthpiece comprises an upper retainer or lower retainer.

9. The device of claim 7, wherein the mouthpiece comprises a palatal overlay or a gingival overlay.

10. A tongue advancement device, comprising:
a mouthpiece configured to fit within the mouth of a user such that the mouthpiece fits over at least a portion of the teeth of the user;
at least one first magnet provided in the mouthpiece;
a tongue cover comprising a flexible tubular body having an opened end and a closed end, the opened end is configured to receive the tongue such that the flexible tubular body encircles and conforms to the shape of the tongue when inserted therein, and at least a portion of the tubular body includes a permeable surface to enable fluids to pass through and contact the tongue; and
at least one second magnet provided at the closed end of the tongue cover to magnetically interact with the at least one first magnet to retain the tongue of the user in a forward position.

11. The device of claim 10, wherein the permeable surface comprises a plurality of perforations.

12. The device of claim 10, wherein the tongue cover comprises a tongue sleeve including a perforated surface.

13. The device of claim 10, wherein the tongue cover comprises a mesh network configuration.

14. The device of claim 10, wherein the tongue cover comprises a tongue gripper configured having a plurality of cords configured to form a Chinese finger trap configuration and the magnetic interaction between the at least one first magnet and the at least one second magnet generates tension within the plurality of cords to retain the tongue in the forward position.

15. The device of claim 14, wherein removal of the tongue from the tongue gripper is performed by detaching the at least one second magnet from the at least one first magnet to release the tension within the plurality of cords.

16. The device of claim 10, wherein the at least one second magnet is removably attached to the tongue cover such that the at least one second magnet is interchangeable with another magnet having a different magnetic strength.

17. The device of claim 10, wherein the tongue cover comprises a pocket including an opening for removably inserting the at least one second magnet therein to enable the user to selectively retain the tongue at one of the different locations of the magnets.

18. The device of claim 10, wherein the at least one second magnet comprises a plurality of magnets provided in the tongue cover at different locations within the tongue cover.

19. The device of claim 10, further comprising a fastening mechanism with straps attached at the open end for assisting with securing the tongue cover on the tongue of the user.

20. The device of claim 19, wherein the fastening mechanism comprises ear straps, a headband or an adjustable headband.
